(19) United States
(12) Patent Application Publication
(10) Pub. No.: US 2002/0002179 A1
(43) Pub. Date: Jan. 3, 2002

(54) TERAZOSIN IN THE CHRONIC TREATMENT OF THE URINARY SYMPTOMS ASSOCIATED WITH BENIGN PROSTATIC HYPERPLASIA

(76) Inventors: HARRIET N. GLASSMAN, LAKE FOREST, IL (US); DAVID C. JORDAN, LIBERTYVILLE, IL (US); HERBERT LEPOR, MEQUON, WI (US); ROBERT R. LUTHER, LOS ALTOS HILLS, CA (US)

Correspondence Address: ABBOTT LABORATORIES, DEPT. 377 - AP6D-2, 100 ABBOTT PARK ROAD, ABBOTT PARK, IL, 60064-6050, US

(21) Appl. No.: 08/068,878

(22) Filed: May 28, 1993

(51) Int. Cl. A61K 31/40; A01N 43/38

(52) U.S. Cl. 514/310

(57) ABSTRACT

Once daily administration of between 1 mg and 20 mg of 1-(4-amino-6,7-dimethoxy-2-quinazolinyl)-4-[(tetraydro-2-furanyl)carbonyl]piperazine (terazosin) or a pharmaceutically acceptable salt thereof provides a safe and effective means for the chronic treatment of the urinary symptoms associated with benign prostatic hyperplasia. Treatment with terazosin resulted in about 43% reduction in symptoms and a 26% increase in peak urine flow rates compared with baseline.

### TECHNICAL FIELD

The present invention relates to medical methods of treatment. More particularly, the present invention concerns the use of 1-(4-amino-6,7-dimethoxy-2-quinazolinyl)-4-[(tetrahydro-2-furanyl)carbonyl]piperazine (terazosin) in the chronic treatment of prostatism.

### BACKGROUND OF THE INVENTION

Benign prostatic hyperplasia (BPH) refers to the benign overgrowth of the stromal and epithelial elements of the prostate associated with aging. Typically, BPH begins at an age in the mid-fifties and is the most common cause of urinary tract symptoms of men of this age. BPH is apparently extremely rare in men prior to age 40, but at age 60, approximately 50% of men have histological evidence of BPH. The prevalence of BPH continues to increase with age until at age 80, approximately 80% of men have pathological evidence of hyperplasia.

Although prostatic hyperplasia is a common finding in older men, the presence of urinary symptoms is the essential feature that distinguishes simple anatomic enlargement of the prostate from prostatism, which is the clinical syndrome whereby the patient experiences significant obstruction of urinary flow and often a progressive increase of urgency and urinary frequency. It is not uncommon for older men to have a palpably enlarged prostate without showing the symptoms of prostatism. From the patient's perspective, the incidence and progression of urinary symptoms are more important than the mere physical presence of an enlarged prostate.

The discovery in the 1970's (M. Caine, et al., Brit. J. Urol., 47:193-202 (1975)) of large numbers of α-adrenergic receptors in the smooth muscle of the prostatic capsule, and bladder neck led to the conclusion that there is both a static and a dynamic component to bladder outlet obstruction associated with benign prostatic hyperplasia. The static component derives from progressive hyperplasia of the prostate with aging, leading to urethral narrowing, causing symptoms of urinary obstruction. Superimposed on this essentially mechanical problem is the variable degree of smooth muscle contraction controlled by the sympathetic nervous system and which is affected by factors such as stress, cold, and sympathomimetic drugs. It is this dynamic component which explains the often rapid fluctuations in symptoms observed in patients with prostatism.

Following the discovery of large numbers of α-adrenergic receptors in the smooth muscle of the prostatic capsule, and bladder neck, it was suggested that α-adrenergic blocking agents should be useful in the treatment of prostatism. Early studies with phenoxybenzamine, a non-selective α-adrenergic blocker, showed efficacy but widespread use of this compound for that purpose was hampered by the finding that it also possessed significant toxicity. With the introduction of prazosin, a selective α-adrenergic blocker, efficacy comparable to that of phenoxybenzamine had been demonstrated, but with fewer side effects.

The treatment of prostatism with α-adrenergic blocking agents affects only the dynamic component of the condition and does not affect prostate size and the associated static component. As a result, a treatment regimen involving the use of α-adrenergic blocking agents is chronic and on-going during the lifetime of the patient or, at the very least, during the period that urinary symptoms associated with the patient's benign prostatic hyperplasia persist. There is thus a need for a treatment method with an α-adrenergic blocking agent which 1) maintains its effectiveness in treating the urinary symptoms of BPH over long periods of administration, 2) possesses the requisite safety for long-term administration, and 3) can be administered once daily to insure patient compliance and convenience.

### SUMMARY OF THE INVENTION

It has been found as a result of long-range placebo-controlled trials with 996 subjects (636 on terazosin, 360 on placebo) that once-daily administration of 1-(4-amino-6,7-dimethoxy-2-quinazolinyl)-4-[(tetrahydro-2-furanyl)carbonyl]piperazine (terazosin) to male patients with urinary symptoms associated with benign prostatic hyperplasia (i.e. prostatism) improved peak urine flow rates and urinary symptoms. The onset of effect in the trials was rapid, with statistically significant improvement of symptoms compared with placebo measured as early as two weeks after initiation of treatment. Significant improvements in peak urine flow rates compared to placebo occured as early as six weeks after initiation of treatment. The results from an on-going long-term study (494 subjects) also indicate that, at the time of filing of this application, the beneficial effects of terazosin are maintained for treatment periods of over two and one-half years. On the average, treatment with terazosin resulted in about 46% reduction in symptoms and a 30% increase in peak urine flow rates compared with baseline.

Safety data for the once-daily administration of doses of terazosin ranging between 1 mg and 20 mg to male human patients with BPH indicated that the drug is safe and well-tolerated by most patients.

The present invention thus provides a method for the chronic or long-range treatment of the urinary symptoms associated with benign prostatic hyperplasia (prostatism) comprising administering to a male human patient in need of such treatment a therapeutically effective amount of 1-(4amino-6,7-dimethoxy-2-quinazolinyl)-4-[(tetrahydro-2-furanyl)carbonyl]piperazine or a pharmaceutically acceptable salt thereof.

### BRIEF DESCRIPTION OF THE DRAWING

In the drawing:

FIG. 1 is a graph showing the mean change from baseline values of Boyarsky Symptom Index scores for patients with urinary symptoms associated with BPH treated with terazosin over a 30-month period in accordance with the present invention. The change from baseline values of irritative Boyarsky symptom scores are shown by the data line depicted with closed circles, the change from baselines values of obstructive symptom scores are shown by the data line depicted by closed squares, and the change from baseline values of overall symptom scores are shown by the data line depicted by closed triangles.

FIG. 2 is a graph showing the mean increase over baseline of peak urinary flow rates for patients with urinary symptoms associated with BPH treated with terazosin over a 30-month period in accordance with the present invention.

### DETAILED DESCRIPTION

As used throughout this specification and the appended claims, the term “chronic treatment” means continuing, on-going treatment for the duration of urinary symptoms associated with benign prostatic hyperplasia and means a period of treatment of at least two and one-half years.

The term “prostatism” means the clinical syndrome caused by the enlargement of the prostate gland by which the patient experiences significant obstruction to urinary flow and often a progressive increase in urgency and urinary frequency.

By the term “pharmaceutically acceptable salt” it is meant those salts which are, within the scope of sound medical judgement, suitable for use in contact with the tissues of humans without undue toxicity, irritation, allergic response and the like, and are commensurate with a reasonable benefit/risk ratio. Pharmaceutically acceptable salts are well known in the art. For example, S. M Berge, et al. describe pharmaceutically acceptable salts in detail in J. Pharmaceutical Sciences, 1977, 66:1-19. Representative acid addition salts of terazosin include the acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. The preferred salt of terazosin for administration in the method of this invention is the hydrochloride.

Pharmaceutical compositions of terazosin for administration in the method of this invention may be specially formulated for oral administration in solid or liquid form, for parenteral injection, or for rectal administration. Preferred dosage forms are oral dosage forms, particularly tablets, capsules, or soft elastic gelatin capsule forms containing unit doses of 1 mg, 2 mg, 5 mg, 10 mg or 20 mg of terazosin.

The pharmaceutical compositions of this invention can be administered to humans orally, rectally, transdermally, bucally, or as an oral or nasal spray. The preferred route of administration is orally by tablet or capsule.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-modifying agents such as sugars, sodium chloride, and the like. Prolonged absorption of the injectable pharmaceutical form may be brought about by the inclusion of agents which delay absorption.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. Soft elastic gelatin capsule (also called “SEC) formulations comprise an outer soft elastic shell of gelatin which is plasticised by the addition of glycerin, sorbitol or other polyol. The shell comprises primarily a gelatin/glycerin mixture ranging between about 50 weight percent gelatin to about 60 weight percent gelatin, with about 55 to 58 weight percent gelatin being preferred. Small amounts of antimicrobial or antifungal agents such as methylparaben, propylparaben and mixtures of the alkylparabens are also present in amounts ranging between about 0.25 weight percent to about 0.4 weight percent, preferably about 0.3 weight percent of the total soft gelatin capsule shell weight. Flavoring agents are optionally added to the soft elastic gelatin capsule formula in amounts ranging between about 0.25 weight percent to about 0.4 weight percent, preferably about 0.3 weight percent of the total soft gelatin capsule shell weight to provide a pleasant taste to the capsule formulation. A preferred flavoring agent for formulations in accordance with the present invention is ethyl vanillin.

Opacifying or coloring agents such as titanium dioxide or iron oxide are also optionally added in amounts ranging between about 0.25 weight percent to about 0.5 weight percent, preferably about 0.3 weight percent of the total soft gelatin capsule shell weight to provide the desired color or opacity to the soft gelatin shell. When white titanium dioxide is used as the opacifying agent, various pharmaceutically acceptable dyes such as FD&C Blue, D&C Yellow, and FD&C Red may also be added to obtain the desired final color of the gelatin shell for identification purposes.

The inner fill composition of soft elastic gelatin capsule formulations comprises from between 1 and 20 mg of 1-(4-amino-6,7-dimethoxy-2-quinazolinyl)-4-[(tetrahydro-2-furanyl)carbonyl]piperazine or a pharmaceutically acceptable salt thereof, with the hydrochloride salt being particularly preferred. The active drug component is dissolved and/or suspended in an inert, non-aqueous, pharmaceutically acceptable liquid carrier. Preferred carriers are selected from the polyethylene glycols, known in the trade as “PEG's,” particularly PEG's having an average molecular weight in the range between about 200 and 600, with PEG 400 being most preferred. The liquid fill comprises from about 80-300 mg of PEG with the typical amount of PEG ranging between about 90-110 mg. The carrier may also include viscosity-building agents such as polyvinylpyrrolidone or silica gel and agents which enhance the suspendability of the drug component in the liquid carrier. Small amounts of glycerin, ranging between about 1-4 weight percent, preferably about 1.5-2 weight percent, based upon the total weight of the fill composition, may also be added to the liquid carrier to retard the migration of glycerin out of the soft elastic gelatin capsule shell into the inner liquid fill.

SEC formulations are generally prepared by methods well known in the formulation arts. See, for example “Remington's Pharmaceutical Sciences,” 18th Edition, A. Gennaro, Ed., Mack Publishing Co., Easton, Pa., 1990, pp. 1662-1663. The capsules may be made by the so-called “plate process” in which a warm sheet of plasticized gelatin is placed on one of two molds containing depressions into which the drug fill is placed. A second sheet of warm gelatin and the second half of the mold is placed over the first and the sandwich pressed to form the soft elastic capsules. This technique permits the formation of soft elastic capsules in which the two halves of the capsule may be of different color for identification purposes.

In the so-called rotary-die process, two ribbons of gelatin are fed continuously into a rotating die assembly which converge adjacent to a fill-injector. The two gelatin ribbons are simultaneously formed into the two halves of the capsule which are filled and subsequently sealed as the die assembly rotates. The fill-injector is actuated by a pump which measures and dispenses the appropriate volume (dose) of the active drug component. This process permits accurate and reproducible formulations.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the abovementioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Compounds of the present invention can also be administered in the form of liposomes. As is known in the art, liposomes are generally derived from phospholipids or other lipid substances. Liposomes are formed by mono- or multi- lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic.

Methods to form liposomes are known in the art. See, for example, Prescott, Ed., Methods in Cell Biology, Volume XIV, Academic Press, New York, N.Y. (1976), p. 33 et seq.

In the treatment method of this invention, terazosin is administered in single daily doses ranging between about 1 mg up to the maximum of 20 mg for a patient of about 55 kg body weight. The dose for a given patient will, however, vary depending upon the weight and physical condition of the patient, his prior medical history, and the severity of the patient's condition. However, it is within the skill of the art for the physician to determine the effective dose in a particular instance by “titrating” the dose; that is, by beginning treatment with a lower dose than is necessary to produce the desired therapeutic effect and gradually increasing the dose until the desired effect is achieved.

Safety and Efficacy of Terazosin in the Long-Term Treatment of Prostatism

A long-range multicenter, open-label study currently designed to last four years and to test the safety and efficacy of the treatment of prostatism with terazosin is in progress at the time of filing of this application. Data from patients who have participated in the study up to two and one-half years are currently available.

Patients in the study are age forty years or older, and have a urinary obstructive symptom scores on the Boyarsky Symptom Index of four or greater based upon the sum of two lead-in visits, peak urinary flow rates of less than 15 mL/second, voided urinary volumes of greater than 125 mL and residual urinary volumes of less than or equal to 200 mL prior to treatment with terazosin. Patients with evidence of prostate cancer were excluded from the study.

Patient's doses were titrated to final doses of 1 mg, 2 mg, 5 mg, 10 mg or 20 mg of terazosin and patients were maintained at that dose level. Final dose levels were determined to be the dose at which response was observed in a given patient or the dose being currently administered when the patient withdrew from the study.

The primary measures of efficacy in these studies were changes in the peak urine flow rate and urinary symptom scores on the Boyarsky Symptom Index. Peak flow rates and voided urine volumes were determined in the standing position, approximately twenty-four hours after dosing using a Dantec Urodyn 1000 uroflowmeter. A minimum voided urine volume of 125-150 mL was required for a valid urine flow rate determination. Peak flow rates and voided volumes were recorded electronically for each determination.

Urinary symptoms were assessed using the interviewer-administered Boyarsky Symptom Index (S. Boyarsky, et al., “A New Look at Bladder Neck Obstruction by the Food and Drug Administration Regulators: Guidelines for Investigation of Benign Prostatic Hypertrophy,” Trans, Amer, Assoc, Genito-Urinary Surg., 68:29 (1977). The Boyarsky Symptom Index includes five questions relating to the patient's obstructive BPH symptoms and four questions relating to irritative symptoms of BPH. A Boyarsky Index rating of zero indicates that a given symptom is minimal or absent and an Index rating of three reflects a severe or frequent symptom. Obstructive urinary symptoms associated with BPH include urinary hesitancy, intermittency, terminal dribbling, impairment in the size and force of the urinary stream, and the sensation of incomplete emptying of the bladder. Irritative symptoms include daytime frequency of urination, nocturia, a sensation of urgency, and dysuria.

Prior to initiation of the clinical trials, experts in urology were consulted regarding what, in their estimation, would constitute a clinically meaningful change in symptom scores and peak flow rates. On the recommendation of these experts, a clinically important change in either variable was defined as a 30% improvement over baseline.

Table 1 shows the distribution of final doses in the 494 patients for which data were available at the time of filing of this application.

TABLE 1

| | Number of |
|---|---|
| Final Dose | Patients |
| 1 mg/day | 38 |
| 2 mg/day | 59 |
| 5 mg/day | 131 |
| 10 mg/day | 166 |
| 20 mg/day | 100 |

The baseline characteristics of the patient population for this study are shown in Table 2.

TABLE 2
Baseline Characteristics of Patient Population

| Baseline Characteristic | Mean (SE*) |
|---|---|
| Age (years) | 63.2 ± 0.3 |
| Prostate size (grams) | 41 ± 1.3 |
| Boyarsky Symptom Index score | |
| Total score | 10.4 ± 0.2 |
| Obstructive score | 6.1 ± 0.1 |
| Irritative score | 4.3 ± 0.1 |
| Urinary flow rate (mL/sec) | |
| Peak | 10.0 ± 0.1 |
| Mean | 5.1 ± 0.1 |
| Voided volume (mL) | 250 ± 4.8 |
| Post-voided residual volume (mL) | 87 ± 4.0 |

*SE = Standard error of the mean

Table 3 shows the duration of treatment for the patient population in this on-study.

TABLE 3
Duration of Treatment

| Interval | Number of Patients (%) |
|---|---|
| ≦90 Days | 35 (7%) |
| 91-180 Days | 44 (9%) |
| 181-360 Days | 79 (16%) |
| 361-540 Days | 72 (15%) |
| 541-720 Days | 93 (19%) |
| 721-900 Days | 129 (26%) |
| >900 Days | 42 (9%) |
| Total | 494 |

The mean changes in Boyarsky Symptom Index scores, peak urinary flow rates, and residual urinary volume are shown in Tables 4,5, and 6, respectively.

TABLE 4
Mean Changes in Boyarsky Symptom
Index Scores

| | | Obstructive | | Irritative | | | |
|---|---|---|---|---|---|---|---|
| Patient | | Symptom | | Symptom | | Total Symptom | |
| Population | | Score | | Score | | Score | |
| Treatm't | | Mean | | Mean | | Mean | |
| Length | No. of | Base- | Mean | Base- | Mean | Base- | Mean |
| (Mo.) | Patients | line | Change | line | Change | line | Change |
| 1 | 474 | 6.1 | −1.5 | 4.3 | −0.5 | 10.5 | −2.1 |
| 2 | 450 | 6.2 | −2.3 | 4.3 | −0.9 | 10.5 | −3.2 |
| 3 | 457 | 6.1 | −3.0 | 4.3 | −1.1 | 10.4 | −4.1 |
| 6 | 428 | 6.1 | −3.3 | 4.3 | −1.2 | 10.4 | −4.5 |
| 9 | 379 | 6.1 | −3.4 | 4.4 | −1.4 | 10.5 | −4.7 |
| 12 | 347 | 6.1 | −3.5 | 4.3 | −1.2 | 10.4 | −4.8 |
| 15 | 281 | 6.1 | −3.8 | 4.2 | −1.2 | 10.3 | −5.0 |
| 18 | 263 | 6.1 | −3.7 | 4.2 | −1.1 | 10.3 | −4.9 |
| 21 | 231 | 6.3 | −4.0 | 4.2 | −1.3 | 10.5 | −5.2 |
| 24 | 207 | 6.2 | −3.8 | 4.2 | −1.2 | 10.4 | −5.0 |
| 27 | 96 | 6.6 | −3.8 | 4.2 | −1.2 | 10.7 | −5.0 |
| 30 | 64 | 6.7 | −4.0 | 4.6 | −1.3 | 11.3 | −5.3 |

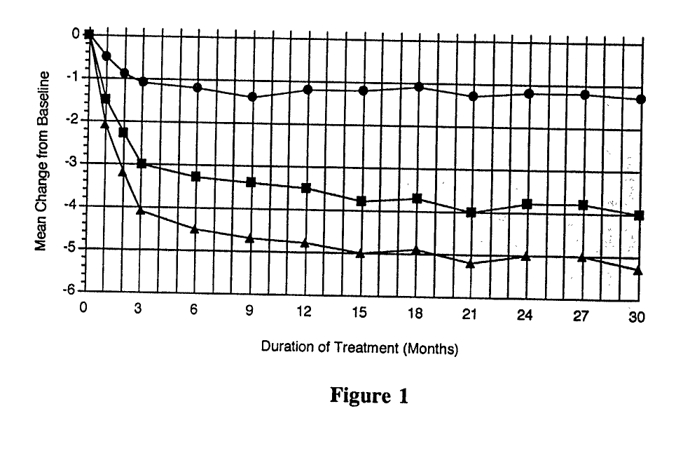

The data appearing in Table 4 have been plotted in FIG. 1 where the change from baseline values of irritative Boyarsky symptom scores are shown by the data line depicted with closed circles, the change from baseline values of obstructive symptom scores are shown by the data line depicted by closed squares, and the change from baseline values of overall symptom scores are shown by the data line depicted by closed triangles.

The data show a significant and sustained decrease in all symptom scores over the 30-month treatment period for which data are available at the time of filing of this application.

TABLE 5
Mean Changes in Peak Urinary Flow Rates

| Patient Population | | | |
|---|---|---|---|
| Lenth of | Number of | Peak Urinary Flow Rates | |
| Treatment (Mo.) | Patients | Mean Baseline | Mean Change |
| 1 | 473 | 9.9 | 1.0 |
| 2 | 447 | 9.9 | 1.4 |
| 3 | 457 | 10.0 | 2.3 |
| 6 | 429 | 9.9 | 2.4 |
| 9 | 380 | 9.9 | 2.6 |
| 12 | 351 | 9.8 | 2.3 |
| 15 | 283 | 9.8 | 2.6 |
| 18 | 264 | 9.8 | 2.8 |
| 21 | 233 | 9.8 | 3.1 |
| 24 | 206 | 9.9 | 2.8 |
| 27 | 97 | 9.0 | 2.9 |
| 30 | 64 | 9.3 | 3.1 |

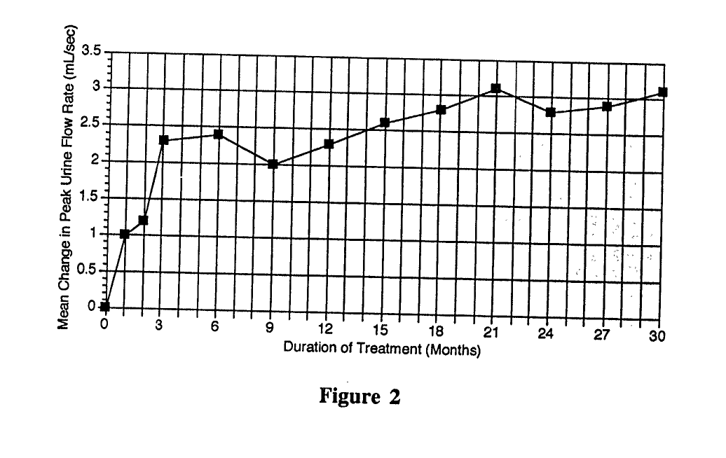

The data from Table 5 have been depicted graphically in FIG. 2 which show a significant sustained increase in peak urinary flow rate in patients treated with terazosin over the 30-month period for which data are available at the time of filing of this application.

TABLE 6
Mean Changes in Residual Urinary Volume

| Patient Population | | | |
|---|---|---|---|
| Lenth of | Number of | Mean Residual Volume (mL) | |
| Treatment (Mo.) | Patients | Mean Baseline | Mean Change |
| 6 | 324 | 89.3 | −11.8 |
| 12 | 304 | 90.0 | −2.9 |
| 18 | 220 | 86.1 | −13.2 |
| 24 | 160 | 86.9 | −4.3 |
| 30 | 34 | 84.3 | −5.1 |

The data in Tables 4-6 above illustrate a beneficial effect of treatment with terazosin of prostatism which is maintained over an extended period of treatment. For example, in Table 4 it can be seen that after 30 months of once-daily treatment with terazosin, the overall Boyarsky Symptom Index of treated patients had been decreased by approximately 46%. Peak urine flow rates, as shown in Table 5, showed an increase of about 30% even after 30 months of treatment.

We claim:

1. A method for the chronic treatment of urinary symptoms associated with benign prostatic hyperplasia comprising administering to a male human patient in need of such treatment a therapeutically-effective amount of 1(4-amino-6,7-dimethoxy-2-quinazolinyl)-4-[(tetrahydro-2-furanyl)-carbonyl]piperazine or a pharmaceutically-acceptable salt thereof.

2. A method for the chronic treatment of urinary symptoms associated with benign prostatic hyperplasia comprising administering to a male human patient in need of such treatment a therapeutically-effective amount of 1-(4-amino-6,7-dimethoxy-2-quinazolinyl)-4-[(tetrahydro-2-furanyl)-carbonyl]piperazine monohydrochloride dihydrate.

3. The method of claim 1 wherein said therapeutically-effective amount comprises a total daily dose ranging between about 1 mg to about 20 mg of 1-(4-amino-6,7-dimethoxy-2-quinazolinyl)-4-[(tetrahydro-2-furanyl)carbonyl]piperazine or a pharmaceutically-acceptable salt thereof.

4. The method of claim 2 wherein said therapeutically-effective amount comprises a total daily dose ranging between about 1 mg to about 20 mg of 1-(4-amino-6,7-dimethoxy-2-quinazolinyl)-4-[(tetrahydro-2-furanyl)-carbonyl]piperazine monohydrochloride dihydrate.

5. The method of claim 3 wherein said therapeutically-effective amount comprises a total daily dose ranging between about 5 mg to about 10 mg of 1-(4-amino-6,7-dimethoxy-2-quinazolinyl)-4-[(tetrahydro-2-furanyl)-carbonyl]piperazine or a pharmaceutically-acceptable salt thereof.

6. The method of claim 4 wherein said therapeutically-effective amount comprises a total daily dose ranging between about 5 mg to about 10 mg of 1-(4-amino-6,7-dimethoxy-2-quinazolinyl)-4-[(tetrahydro-2-furanyl)carbonyl]piperazine monohydrochloride dihydrate.

7. The method of claim 1 wherein said therapeutically effective amount of 1-(4-amino-6,7-dimethoxy-2-quinazolinyl)-4-(2-tetrahydrofuroyl)piperazine or a pharmaceutically acceptable salt thereof is administered in the form of a soft elastic gelatin capsule formulation in unit dosage form comprising between 1 mg and 20 mg of active ingredient.

8. The method of claim 1 wherein said 1-(4amino-6,7-dimethoxy-2-quinazolinyl)-4-[(tetrahydro-2-furanyl)carbonyl]piperazine or a pharmaceutically acceptable salt thereof is administered to a human male patient for a period of at least two and one-half years.
